(19) United States
(12) Patent Application Publication
(10) Pub. No.: US 2005/0192528 A1
(43) Pub. Date: Sep. 1, 2005

(54) Methods, apparatus and charged chemicals for control of ions, molecules or electrons

(76) Inventor: Robert Tapper, Los Angeles, CA (US)

Correspondence Address: FULWIDER PATTON LEE & UTECHT, LLP;HOWARD HUGHES CENTER, 6060 CENTER DRIVE, TENTH FLOOR, LOS ANGELES, CA, 90045, US

(21) Appl. No.: 11/029,904

(22) Filed: Jan. 4, 2005

Related U.S. Application Data
(60) Provisional application No. 60/535,470, filed on Jan. 8, 2004.
(60) Provisional application No. 60/543,446, filed on Feb. 9, 2004.
(60) Provisional application No. 60/593,030, filed on Jul. 29, 2004.

(51) Int. Cl. A61N 1/30

(52) U.S. Cl. 604/20; 131/352; 424/447; 424/65

(57) ABSTRACT

A system including methods, apparatus, components and charged chemicals for control of ions, molecules or electrons whereby charged membranes, testing devices, electrode patch structures and the like utilize features of the invention for control of flow in a wide variety of new and improved medical, testing, cosmetic, personal care, flow delivery applications and the like.

### CROSS-REFERENCE TO RELATED APPLICATION

This application is claiming the benefit of a co-pending provisional application Ser. No. 60/535,470 filed on Jan. 8, 2004, co-pending Ser. No. 60/543,446 filed on Feb. 9, 2004 and co-pending Ser. No. 60/593,030 filed on Jul. 29, 2004. The material of the related provisional applications are incorporated by reference herein.

### FIELD OF THE INVENTION

This invention relates generally to methods and apparatus pertaining to charged particles flow and, more particularly, to improvements in methods, apparatus, systems and materials for control of flow and level of ions, molecules, or electrons using charged chemicals and any/all applications thereof.

### BACKGROUND OF THE INVENTION

Topical drug delivery systems range from small particulate carriers through passive patches to sophisticated iontophoretic propulsion delivery systems. Ideally, they attempt to deliver beneath the skin beneficial chemicals or drugs in the largest controlled amounts, in the shortest time, up to the largest molecular size and without chemically caused skin injury or microneedle puncture. No commercially available product can do all of this today. Accordingly, those of ordinary skill in the art have long recognized the need for improvements in these areas, and the present invention fulfills all of these needs.

### INVENTION SUMMARY

Basically, the present invention satisfies the aforementioned needs with improvements in methods, apparatus, components and chemistry including the use of charged chemicals of either polarity or both as chemically integral surfaces on support membranes or equivalent support materials such as felt or like materials made of natural or synthetic fibers or impregnated filter paper or other materials in a drug delivery or diagnostic withdrawal system. The charged chemicals may also be used without a support structure. They allow multiple hybridizations that could include a neutral charge among other effects. Examples of these membranes manufactured by Pall Corporation of 25 Harbor Park Drive, Port Washington, N.Y. 11050 are Mustang S, Mustang Q, Mustang C and Biodyne A, Biodyne B and Biodyne C. The Mustang series are of special interest with Mustang S giving a strong negative polarity with its surface modified by sulfonic acid. Mustang Q gives a strong positive polarity with its surface modified by quaternary amine. It should be understood that the chemicals cited are for example only and are not the only chemicals that can polarize support structures or control ion or molecule flow without a support structure. For instance, Mustang C is polarized with carboxylic acid. Other examples of functional groups that can bear a charge would be the hydroxyl, phosphate moieties. A unique feature of the invention is the use of charged or polarized chemicals on support members or not, to control a flux, current, or signal of polarized molecules, ions or electrons and even polar neutral compounds as in an electroosmotic withdrawal system and a drug delivery system.

The invention is contemplated, in its various forms and applications as including the following and other features:

1) The use of charged chemicals of either negative or positive polarity on support members that could include a) membranes, b) felt pads made of natural or synthetic fibers, c) impregnated filter paper, d) liquid form, e) any material that allows charged chemicals to control ions, molecules or electrons;

2) The use of charged chemicals of either negative or positive polarity formulated with an increased concentration of the charged chemicals causing either negative or positive polarity to increase their effectiveness;

3) For use in a DC iontophoretic drug delivery system, the presence of charged chemicals in solution as an integral part of a felt pad(s) or a membrane(s) to prevent: a) injurious chemicals emanating from the electrode from reaching the skin, b) sodium hydroxide developed at the negative terminal is prevented from reaching the skin with either a negatively charged or positively charged intervenor between the skin and the electrode in an electrically conductive circuit, c) hydrochloric acid generated at the positive electrode can be prevented from reaching the skin with either positively charged or negatively charged chemicals on an appropriate support intervenor spaced between the electrode and the skin in an electrically conductive system;

4) The chemically charged intervenor(s) acting as a reservoir or storage area for the drug to be delivered;

5) In iontophoresis or reverse iontophoresis or drug delivery or similar chemical or drug transport system, the use of currents above the traditional 0.5 ma per cm². The aforementioned charged chemicals either on support structure or without support structure, enable these high currents to be achieved. Large molecular delivery also benefits from high electrical current along with Tapper U.S. Pat. Nos. 6,238,381 and 6,425,891;

6) In a powered patch, electrical currents can be further increased by a new physical configuration of the active, drug delivery applicator. High density current can be tolerated when multiple small circles of current emitting membranes are clustered instead of one large flat delivery surface;

7) The use of the aforementioned charged chemicals of both negative and positive polarity together or separately to meet all objectives of these inventions;

8) The use of charged chemicals on an intervenor or in combination with both polarities in an AC iontophoretic device;

9) The use of histamines in the positive applicator to lessen the pain from this applicator and allow an increase in current;

10) The use of charged membrane(s) in an otherwise unpowered patch (passive patch) to propel the drug(s) into the skin at greatly increased levels compared to other unpowered patches;

11) The use of charged membrane(s) in an unpowered patch as a storage area or reservoir for drugs.

12) The use of charged membrane(s) of either polarity or in combination to increase infusion in an otherwise unpowered patch;

13) The use of charged membranes between skin and output electrode as the conductive element when wetted with distilled water without the need of a saline solution;

14) The use in iontophoresis or reverse iontophoresis of charged membranes between the skin and output electrode as the conductive element when wetted with distilled water as a means of avoiding clutter from conductive chemicals that may be added to enhance transport;

15) The use of charged membrane(s) in a powered patch of either polarity or in combination as an intervenor to prevent skin injury;

16) The use of charged membrane(s) in a powered patch of either polarity or in combination to act as a drug storage area or reservoir;

17) The use of charged membrane(s) in a powered patch of either polarity or in combination as a reservoir and intervenor to increase currents above 0.5 ma per cm² without skin injury;

18) In drug delivery, a small amount of glucose (0.2 mol/l) in solution with insulin can be amplified 9 times in an unpowered environment using charged membranes;

19) Feature 18 wherein the above solution is in a powered environment will increase the signal many-fold;

20) Feature 19 in a powered environment with the use of polarized membranes to act both to prevent skin injury and further enhance amplification;

21) In drug delivery, any chemical or drug in a powered environment and stored in oppositely charged membrane(s) will cause amplification;

22) Another example of drug or chemical amplification is using the phenomena of oppositely charged drugs to create recycling and thus amplification would be with glucosamine and chondroitin. Glucosamine is positive and chondroitin is negative and the two in solution with the appropriately charged membranes and in an appropriately polarized field, would benefit from amplification;

23) The use of charged chemicals on filters in cigarettes positioned between the tobacco and the end held in the mouth to prevent the migration of deleterious tobacco chemicals from entering the mouth upon inhaling;

24) The use of charged chemicals of either polarity or in combination on impregnated filter paper or membranes as an intervenor between tobacco and mouth to prevent polarized harmful chemicals from reaching the mouth.

25) The use of a tobacco extract or flavor in combination with Items 23 and 24;

26) A charged membrane of the same polarity of the tobacco extract or flavor to propel the extract or flavor into the mouth;

27) The use of charged chemicals as an inherent part of a bandage or the like, to have an antibacterial effect when placed over a wound;

28) The use of a chemically charged bandage or the like that comes precoated with an antiseptic or the antiseptic is added later. The charged chemicals will drive the antiseptic into the wound continuously when wetted or in gel form for communication between all elements;

29) The use of a chemically charged bandage or the like to enhance and speed wound healing when wetted;

30) The use of a charged chemical of negative polarity in a toothpaste containing fluoride to infuse the fluoride below the tooth's surface and gum to prevent cavities and disease;

31) The use of a charged chemical of negative polarity as an integral part or coating of toothbrush bristles to cause the fluoride of a toothpaste to be driven or infused into the teeth or gums;

32) The use of charged chemicals of either polarity as an additive to a germ-killing mouthwash to infuse the antiseptic into the teeth and gums;

33) The use of a charged chemical of negative polarity to be used as an intervenor between a battery-powered iontophoretic toothbrush and the bristles to prevent injurious sodium hydroxide from the negative terminal of the battery from reaching the teeth, gums or mucous membrane;

34) A stent coated with either positive or negative chemicals or both to cause elution and prevent restenosis.

35) A stent with a coating of charged chemicals of either charge or both that will elute like-charged chemicals coated on top of the charged coating to prevent restenosis;

36) A stent with a coating of charged chemicals either positively or negatively charged or both integrated with like-charged chemicals that will be propelled or eluted from the surface to prevent restenosis;

37) A stent coated with quaternary amine, sulfonic acid or carboxyl acid or the equivalent to cause elution either with another chemical that will be driven beyond the surface or the charged coating alone to elute when surrounding tissue and body fluids come in contact;

38) For cosmetic application, the use of charged chemicals in liquid form as a spray to be applied over a moisturizer base or any other skin conditioner. This will drive a like-charged cosmetic ingredient or skin improvement product deeper into the skin than topical application;

39) The use of salicylic acid and/or its derivative in combination with a charged chemical to limit its travel beneath the skin and thus prevent irritation;

40) The use of positively charged salicylic acid in combination with negatively charged sulfonic acid or carboxyl acid or the like to bind to each other and thus limit migration of the salicylic acid beneath the epidermis;

41) The use of salicylic acid as a skin spray as part of a two component system wherein a negatively charged spray follows;

42) Salicylic acid's pH buffered from three to four to a nonirritating pH of approximately five and infused into the skin by a like-charged chemical;

43) The use of negatively charged sodium salicylate with negatively charged chemicals to infuse the sodium salicylate into the skin for beneficial effects. The sodium salicylate and the negatively charged chemicals may be in formulation or they may be in the form of a two component system whereby the sodium salicylate is applied first as perhaps a spray and followed by a spray of negatively charged chemicals;

44) For cosmetic and personal care application, the use of charged chemicals in formulation with like-charged skin improvement material. The repelled skin improvement materials will be infused deeper into the skin;

45) The use of the negative and positive charges in formulation to result in a neutral charge and then mixed with the salicylic acid to limit its penetration to the epidermis;

46) The neutral formulation of Item 45 to be used as a spray following the application of salicylic acid to limit its penetration;

47) The use of positively charged chemicals such as quaternary amine in solution with salicylic acid to limit the depth of its penetration to the epidermis;

48) The use of charged or neutral control chemicals in the form of a spray to be applied over the initial application of salicylic acid to limit its depth of penetration to the epidermis;

49) In a permanent hair remover (Tapper U.S. Pat. Nos. 6,094,594 and 6,206,869) wherein a depilatory is driven into the follicle by an iontophoretic device, the use of a charged intervenor between battery and skin for the purposes of: a) to use high currents to expedite treatment, b) to use chemically charged intervenor to prevent skin injury, c) to use the chemically charged intervenor as the storage or reservoir vehicle for the depilatory;

50) A non-invasive diagnostic withdrawal device using a charged or polarized membrane(s), one end of which is positioned to touch the skin and the other end touching the electrode;

51) An alternate construction of Item 50 is the use of two membranes, one charged or conductive and the other nonconductive in direct contact with each other and spaced between the skin and the electrode;

52) The use of a charged membrane and an uncharged membrane in a withdrawal system whereby the uncharged membrane is placed in contact with the skin and the charged membrane in contact with the electrode to complete the circuit.

53) The use of a wool felt nib such as from a marker pen as the intevenor between the skin and electrode to prevent the passage of sodium hydroxide from the negative electrode from passing to the skin. The wool nib may or may not be coated with charged chemicals;

54) The charged or polarized intervenor of Item 50 that is approximately a half-inch in length and placed between skin and electrode;

55) The membrane of Items 50-54 between the skin and the electrode to protect against skin injury;

56) Increasing the height of this membrane of Items 50, 41 and 55 allows the use of currents above 0.5 ma per cm²;

57) This charged membrane intervenor of Items 50, 54, 55 and 56 allows the use of high currents above the traditional 0.5 ma/cm²;

58) Increasing the level of charge of the aforementioned membrane(s) by higher concentrations of the polarized material improves the benefits cited above;

59) A positively charged wetted (gel) membrane prevents skin injury by stopping the sodium hydroxide ions emitted from the negative electrode in an electrically conductive circuit;

60) The charged membrane interposed between skin and negative terminal also stores the solution (or gel) necessary to effect communication or current flow between skin and electrode;

61) The target withdrawn glucose analyte passes through the membrane to the end that touches the negative electrode and becomes the critical pick-up point for the glucose monitor to read. This pick-up point for the withdrawn glucose analyte is unique in that it is against the electrode and not at membrane entry point in contact with the skin;

62) The charged membrane is constructed in a rolled form so that one side of the membrane touches the skin and the other side touches the electrode. In other words, when viewed from the skin, the withdrawn analyte sees only straight line, unbroken surfaces while migrating to the electrode;

63) The membrane form of Item 62 that causes the withdrawn glucose analyte to flow in a straight line while migrating to the electrode;

64) The structure of Items 62 and 63 that causes the withdrawn glucose signal deposition on the end of the membrane in direct contact with the electrode;

65) The use of a high pH from the electrode that attracts the glucose to this point of signal deposition;

66) The structure of Features 62, 63, 64 and 65 wherein the membrane end in contact with the electrode is the pick-up point for the withdrawn glucose which is then placed in contact with the monitor's strip for analysis or direct reading;

67) The positive return electrode may also use charged membranes to prevent skin damage and allows toleration of higher currents;

68) The positive return electrode of the system described above may also be used to monitor drug pharmacokinetics;

69) A solution formulated with glucose in a solvent of distilled deionized water;

70) The solution of Item 69 wherein the glucose must totally saturate the distilled deionized water to prevent absorption of the withdrawn glucose;

71) The solution of Items 69 and 70 must have a surplus of glucose (distilled deionized water solvent totally saturated) with a monitor reading between 1 and 400 mg/dl or more;

72) Adding a small quantity of insulin to the glucose solutions of Items 69, 70 and 71, perhaps 0.3% or less, greatly increases the analyte signal or causes amplification;

73) The solution of Items 69, 70, 71 and 72 that may include stabilizers or preservatives;

74) Chemical amplification of the minute subpicomole withdrawn analyte takes place in the environment disclosed. When at least two differently charged substances occupy the same area, they become reagents to a signal passing through. The reagents cause recycling of the signal resulting in amplification. The charged positive membrane in the negative field acts as a reagent to the withdrawn glucose analyte and amplifies it;

75) Another form of this would be the use of a positive and negative membrane adjacent to each other to cause recycling and therefore amplification of analyte;

76) Amplification would take place if all the polarities cited above were reversed;

77) Charged membranes made with higher concentration of charged chemicals will show increased amplification;

78) More presence of charged membrane will increase the reaction and therefore increase amplification;

79) This invention lends itself for the new technology of ‘labs-on-a-chip’;

80) The electrode is a screen made of stainless steel to evenly disperse sodium hydroxide and pH;

81) The power supply consists of a 6 volt battery with circuitry to increase the DC output voltage to 70 volts. Note: voltage may be higher or lower. A safety circuit or fail-safe circuit is included;

82) The circuit includes a dosimetry circuit (Tapper patents) that precisely controls the analyte withdrawal quantity based on time and current;

83) This non-invasive diagnostic device includes a calibration switch;

84) A multi-position switch that selects the withdrawal time/current to match the highs, lows, and in-between time glucose levels of the patient caused by meals, physical exercise, or insulin dose;

85) A multiple position switch that selectively adjusts time and current to conform with well established periods of glucose change related to meal intake (also physical exercise and insulin dose);

86) The switch of Items 84 and 85 for following selections: Position 1 (1-2 hours after meals), Position 2 (2-3 hours after meals), and Position 3 (3-4 hours after meals);

87) Different time/current rates are assigned to each of the three switch positions;

88) A submultiple or multiple of the meter reading to extend range;

89) After gross selection is made with switch according to Items 83, 84, 85, 86, 87 and 88, a precise reading is obtained when the withdrawn glucose specimen is processed at the strip and results in a meter reading based on the glucose concentration or density in the withdrawn interstitial fluid; and

90) An LED to indicate the precise end to the withdrawal process.

These and other objects and advantages of the invention will become apparent from the following more detailed description, when taken in conjunction with the accompanying drawings of illustrative embodiments.

### BRIEF DESCRIPTION OF THE DRAWINGS

FIG. 1 is a perspective view of one embodiment of a passive patch construction in accordance with the present invention;

FIG. 2 is a perspective view of another passive patch constructed in accordance with the invention;

FIG. 3 is a perspective view of still another passive patch constructed in accordance with the present invention;

FIG. 4 is a perspective view of a powered patch construction in accordance with the present invention;

FIG. 5 is a perspective view of another powered patch in accordance with the present invention, shown in place upon the skin;

FIG. 5a is a perspective view

FIG. 5b is a sectional view of a drug delivery device utilizing the present invention and illustrating multiple clustered electrical current emitting membranes;

FIGS. 6a, 6b and 6c are fragmentary perspective views of filter devices embodying features of the invention;

FIG. 7 is an exploded perspective view of a diagnostic probe constructed in accordance with the present invention;

FIG. 7b is a perspective view of a rolled charged membrane embodying the present invention;

FIG. 7c is an enlarged longitudinal view of a probe including a switch and LED; and

FIG. 7d is an enlarged view of the electrical switch for the probe shown in FIG. 7c.

### DETAILED DESCRIPTION OF THE PREFERRED EMBODIMENTS

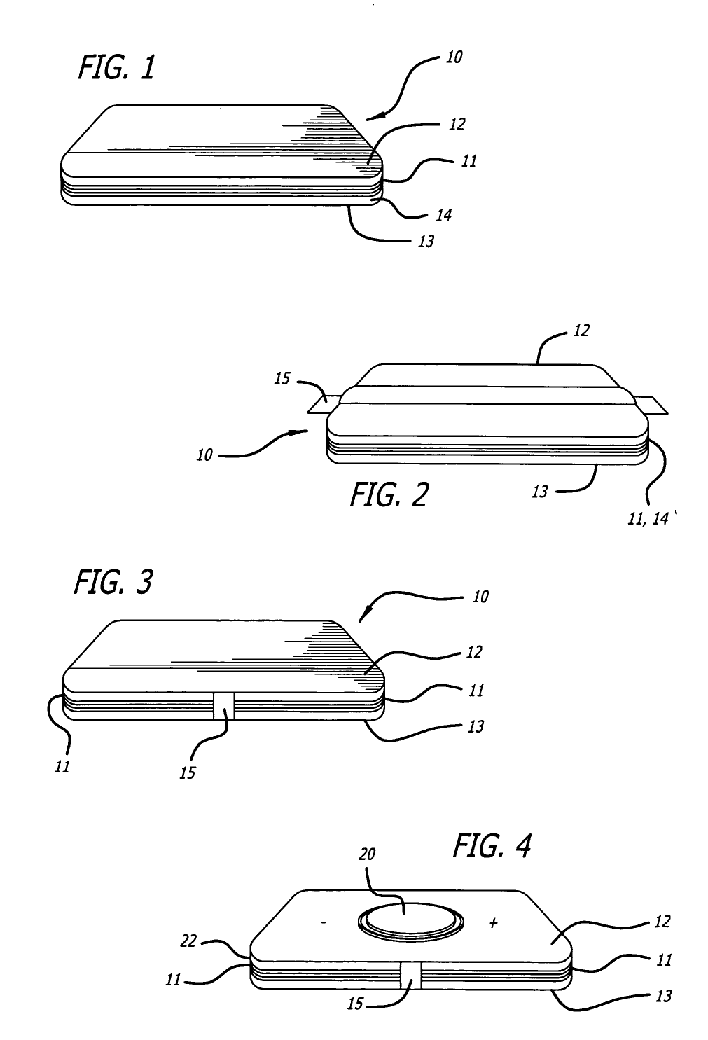

Referring now to the drawings, wherein like reference numerals refer to like or corresponding elements throughout the various figures of the drawings, there is shown in FIG. 1 an improved passive patch 10 embodying features of the present invention. The use of this technology in its simplest form without the use of battery power would be with the commercially available passive patch 10 that is unpowered. The use of one or more polarized membranes 11 beneath a backing 12 acts to propel a drug into the skin if the polarity of the drug (or other chemical) and the membranes 11 were the same. In this instance, a liner 13 would be removed and the charged membranes 11 in communication with a drug-in-adhesive system 14 would propel the drug into the skin (not shown). Another configuration would be for the charged chemicals to be an inherent part of the drug-in-adhesion.

As shown in FIG. 2, the charged membranes 11 also act as a reservoir for the drug 14 with adhesion of the patch 10 made by separate tape 15 or any of a variety of taping means. For the first time, an infusion system without battery, would force much greater quantities of drug (or other chemicals) into the skin. They allow multiple hybridizations that could include a neutral charge among other effects. Examples of these membranes manufactured by Pall Corporation of 25 Harbor Park Drive, Port Washington, N.Y. 11050 are Mustang S, Mustang Q, Mustang C and Biodyne A, Biodyne B and Biodyne C. The Mustang series are of special interest with Mustang S giving a strong negative polarity with its surface modified by sulfonic acid. Mustang Q gives a strong positive polarity with its surface modified by quaternary amine. It should be understood that the chemicals cited are for example only and are not the only chemicals that can polarize support structures or control ion or molecule flow without a support structure. For instance, Mustang C is polarized with carboxylic acid. Other examples of functional groups that can bear a charge would be the hydroxyl, phosphate moieties. A unique feature of the invention is the use of charged or polarized chemicals on support members or not, to control a flux, current, or signal of polarized molecules, ions or electrons and even polar neutral compounds as in an electroosmotic withdrawal system and a drug delivery system.

Referring to FIG. 3 of the drawings, a variation of the patch 10 would be for the inside of the backing 12 to be divided in half by an electrical insulator 15, such as a plastic divider, so that two different medicaments could be placed in each half—one of negative polarity and one of positive polarity with like-polarized membranes in each half to drive the drugs into the skin.

Another variation would be for the patch 10 to have a polarized membranes 11 under the backing 12 with a like-polarized drug saturating it and an opposite charged membrane 11 configured in a circle in close proximity around the inner drug bearing membrane but electrically insulated from it. Over 620 millivolts exist between polarized membranes 11 of one polarity and the skin. This value can be increased by adding more membranes 11 to the stack. It can also be increased by using charged chemicals in greater concentration. Still greater efficiency can be obtained by using charged membranes (or felts, filters, etc.) of both polarities with a membrane battery separator (not shown) in between.

Another very important application of these polarized membranes would be for wound dressing to promote wound healing or infection control. Published papers show that tissue growth is promoted when minor currents traverse the wound. Another paper attributes this growth to the anti-bacterial effect of low currents across a wound. In accordance with the invention, bacterial and fungal infection can be controlled with the electro-chemical effect of charged membranes, bandages or dressing over infected body areas.

A major problem with catheters is their propensity to cause infection at the body insertion opening. Coating or bonding charged chemicals of both polarities separated from each other onto the catheter tubing will give wide spectrum protection against bacterial infection. Medical use of this technology would be for stent coating. The charged coating (either charge or both) would have an eluting effect to prevent restenosis. Another medical use of this technology could include microscopic drug delivery for both external and internal application.

The charged membranes can also infuse an antiseptic for enhanced control. It may also be applied with a spray of the charged chemicals, sulfonic acid or quaternary amine or both or the equivalent. An example of such an application would be to apply a treatment cream or medicament over an area and then spray the polarized chemical over it. If the beneficial formulation applied to the skin is of the same polarity as the charged material on top, the treatment formulas (skin conditioners, etc.) or medicament will be driven into the skin. A variation of this would be to mix the treatment formula of known polarity with a charged chemical of the same polarity and then apply topically to the skin. The like-charged treatment formula will be driven into the skin.

Another example of the practice of this invention is to mix a positively charged chemical with a common “over the counter” antiperspirant. The charged chemical will drive the aluminum chloride or the like deep down the eccrine sweat duct for far more effectiveness than topical application. Another form of this would be a two-component system whereby aluminum chlorhydrate antiperspirant is topically sprayed under the arm and is followed with a spray of a positively charged chemical to infuse the aluminum chlorhydrate deep within the eccrine sweat duct.

Conversely with the above applications wherein there is an effort to drive or infuse the active drug or cosmetic deeper into the skin, there are certain applications where it is desirable to limit the travel of the active components to the epidermis (outer skin). Salicylic acid is an example of this. Salicylic acid and/or its derivative is the treatment of choice for acne, psoriasis, or photoaging. Because of its low pH (3-4), positively charged salicylic acid is capable of penetrating deeply through the epidermis, dermis and receptor fluid causing irritation. To overcome this problem, the use of the negatively charged chemicals of sulfonic acid or carboxyl acid or the like as a control for the permeation of the salicylic acid is suggested. When formulated with the salicylic acid, the negatively charged ions will combine with the positively charged salicylic acid and, when properly balanced, will limit the depth of penetration of the salicylic acid to the epidermis, thus eliminating irritation. Two other combinations using charged chemicals to limit the flow of the active salicylic acid to the epidermis would be the use of the negative and positive charges in formulation to result in a neutral charge. Still another impediment to the salicylic acid flow below the epidermis, would be the use of positively charged chemicals such as quaternary amine in solution with salicylic acid. These charged or neutral control chemicals can be in the form of spray to be applied over the initial application of salicylic acid.

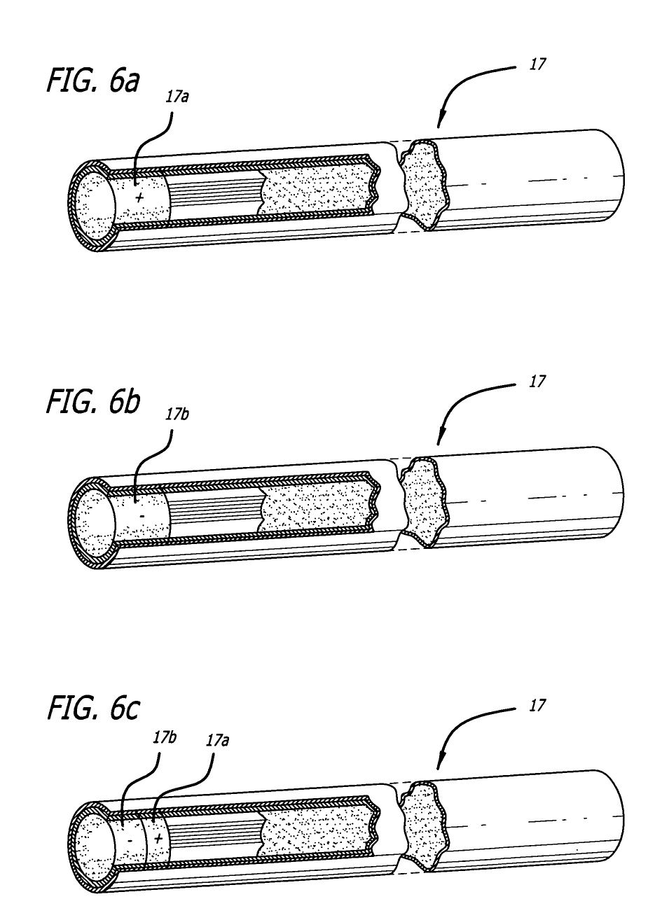

Referring now more particularly to FIGS. 6a, 6b and 6c of the drawings, another major beneficial application of the present invention would be for the use of the positive membrane Mustang Q or a filter paper impregnated with Mustang Q or the like, to repel the positively charged nicotine chemical that is inhaled during smoking to prevent addiction. These membranes or filter paper or the like would take the form of layered filters 17a, 17b, 17c at any location between the lips and the tobacco and will effectively repel nicotine and other harmful chemicals from leaving the cigarette.

Carbon monoxide is another danger to the smoker. Carbon monoxide is formed by the combustion of carbon with a limited supply of air. As best shown in FIG. 6c, a positive membrane or impregnated filter paper 17a in direct contact with a negative membrane or filter paper 17b constitutes a battery and as a result liberates oxygen at the positive membrane and hydrogen at the negative membrane. These added gases would act to neutralize the carbon monoxide. The membranes could be ‘wetted’ with a precoated hydrogel or ‘self-wetting’ since the direct contact between the negative and positive membranes create a ‘moist’ condition. This would be an obvious boon to smokers worldwide since the medical community relates cancer, heart problems, pulmonary problems, etc. to the inhalation of tobacco smoke.

Also contemplated within the purview of the invention is a cigarette filter, as shown in FIG. 6, having a charged chemical(s) which attracts or repels unwanted polarized ions or molecules from a tobacco smoke stream so that they do not enter the mouth. The charged or polarized chemicals (FIGS. 6a, 6b, 6c) are covalently bonded to a non-volatile inorganic substrate which is incorporated in the filter. The filter can remove nicotine, carbon monoxide, cadmium, lead, mercury, nickel, cyanide among other dangerous elements from tobacco smoke. These charged or polarized chemicals can be covalently bonded to silica gel (APS silica gel) or the like. The charged chemical(s) can be contained in a space in the filter or incorporated in one or more filter elements such as tipping paper, shaped paper insert, mouthpiece plug, solid filter element, or free-flow filter element. The charged chemicals can be part of or coated on paper such as tipping or filter paper or incorporated in non-paper filter elements formed from fibrous materials such as cellulose acetate or polypropylene fibers. The use of the charged chemicals on any of the aforementioned exemplary support structures addresses the safety aspect of the invention.

Other aspects of cigarette acceptability involve the tobacco flavor that a smoker seeks for gratification. With the source for this tobacco taste diminished or stopped by the charged membrane(s), it can be restored with the use of tobacco extracts or flavors in the space between the end of the cigarette that goes into the mouth and the membrane or filter in contact with the tobacco. Another variation of this is to coat a charged membrane with the same polarity tobacco extract to propel the extract or flavor into the mouth. The smoker will now experience a cigarette that is safe, imparts flavor, and exudes smoke that is psychologically satisfying.

Another application of the invention having major universal value would be in the field of dentistry. It has long been known that fluoride has beneficial effects for the care and well being of teeth. In this regard, the government has allowed fluoride to be added to the water system. An extension of this is a means of infusing fluoride below the surface of the teeth instead of topical application with a common dentifrice. This has been recognized by the FDA as the ultimate means of preventing cavities and they approved a methodology that uses an iontophoretic toothbrush to infuse a fluoride toothpaste beneath the surface of the tooth. Attempts were made to commercially introduce such a device but it failed at the marketplace. Among the problems is the short-term treatment of a brushing that would have questionable value. If the electrical current were raised, there could be a danger of injury to the very sensitive surrounding mucous membranes and the gum.

With the use of charged chemicals as a stand alone component away from membranes or felt pads, a new application of the present invention presents itself. The negatively charged chemicals (typically sulphonic acid or carboxyl acid and the like) when placed in a mouthwash or dentifrice with the presence of fluoride will act to infuse fluoride beneath the surface of the tooth. In addition, this process could continue over time for the ultimate cavity protection because residual activity may linger for hours. Some high potency fluoride toothpastes are recommended to be used without rinsing. Under these conditions, it is apparent that negatively charged chemicals will repel the negatively charged fluoride over time for the ultimate protection against tooth decay and the prevention of gum disease.

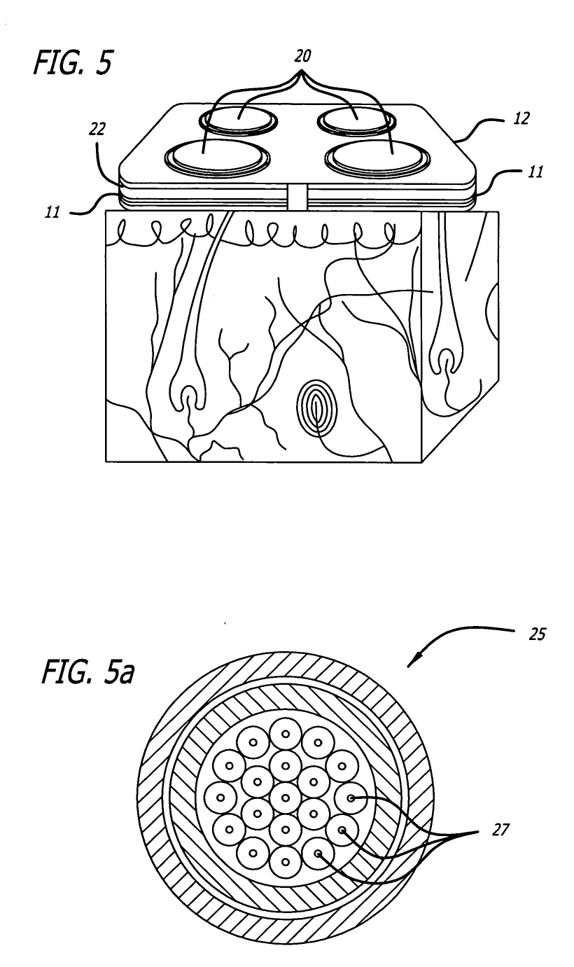

A major advance in noninvasive drug delivery is to introduce a battery 20 (see FIGS. 4 and 5) or any electrical power source to greatly increase fluxes of both ionic and polar neutral compounds. This process known as iontophoresis, works through one or a combination of the following mechanisms: electrophoresis, electroosmosis, and electroporation. While this process was known for over 100 years, its use was extremely limited because of the bums and irritation it could cause, slow treatment since an increase in electrical current would cause unacceptable pain, and an inability to deliver large molecular drugs at therapeutic levels because of low current requirements as well as other needs. All of these handicaps that limit acceptability of a powered superior drug or chemical delivery system are now overcome by means of the present invention directed to the use of charged or polarized membranes.

The following examples of systems using charged membranes with electrically powered systems or battery, presumes the reader is aware that the drug or chemical to be delivered is in solution that could take the form of a liquid, gel or paste or equivalent that links the active elements together. In this instance, the membranes serve the purpose of a reservoir for this solution. The second purpose of the membranes 11 is to protect the skin from injury. Stacked or coiled charged membranes 11 or the equivalent will stop the migration of harmful ions from the battery 20 or electrical power source connected electrodes 22. For instance, multiple Mustang S membranes placed between the negative electrode and the skin will prevent sodium hydroxide from reaching the skin, yet it will allow the flow of electrical current between these two points. Multiple Mustang Q membranes placed between the positive electrode and the skin will prevent hydrochloric acid from reaching the skin. Very importantly, this allows enormous electrical current levels to be used with no skin injury and lessened pain. This means that noninvasive iontophoretic devices can deliver therapeutic levels of drug in the fastest possible time.

Combinations of these membranes 11 may be used under the same electrode 22. For instance, one use of the polarized membrane 11 would be to use the negative membrane against the negative electrode to repel the injury causing sodium hydroxide from reaching the skin. The positive charged membranes could also be used with the negative electrode because the unlike negative ions would bind to the positive membrane and therefore stop the injury causing ions from reaching the skin. Conversely, we may use the negatively charged membrane against the positive electrode to stop the injury causing hydrochloric acid from reaching the skin. The communicating link between electrical power source, through solution wetted membrane to skin, includes the drug or chemical of choice. The solution may even be non-conductive. Conductivity between the power source and the skin is made because the wetted membrane is conductive by virtue of its charged chemicals that electrically link the electrode to the skin. The charged membranes may be renewed and life-extended by simply reversing the polarity of the battery 20 or power source.

The device may be used with an AC signal (as in U.S. Pat. No. 5,224,927). Mixed polarity membranes may be used under each electrode in this instance. Mixed polarity membranes may also be used in a DC system under the same electrode. This has a stabilizing effect and enhances regulation.

A major challenge of this non-invasive technology is the infusion of large molecular drugs such as insulin. Also, the diabetic may need a ‘bolus’ shot (an exceptionally high infusion). Infusion of large molecular drugs requires high electrical currents to drive them into the skin. Another example of the need for high currents to drive extremely high molecular drugs into the skin is the transport of glucosamine and chondroitin. These two chemicals can lessen pain and affect cartilage repair for knee osteoarthritis. Presently, these two chemicals require very large oral administration dosage to be effective. Losses created by first pass metabolism before they reach the area of effectiveness, make it necessary for large oral dosages. Direct application of these drugs to the knee area is superior. The problem arises because negatively charged chondroitin has a molecular weight of 60,000 daltons—over 10 times more than insulin! The aforementioned means of large electrical currents to give high current density are very effective to deliver these large molecules. Skin preps to aid permeability should be used with these drugs for non-invasive delivery. These preps include sodium salicylate and/or depilatories.

In still another effort to increase electrical current, in accordance with the invention, by reconfiguring the traditional flat surface of the active drug-carrying applicator we were able to increase current. Referring to FIG. 5b, a drug-carrying applicator 25 is shown to be multiple small, membrane containing openings 27. As before, the membrane acts as a drug reservoir and as an injury-preventing intervenor between the skin and the source of power. By breaking up a large flat delivery surface, in accordance with the invention, the electrical current could be increased appreciably.

It has been found that another contributor to pain during the iontophoretic process comes from the positive applicator of the device. The cause for the pain and therefore a limiting current factor at the positive output, is believed to be the fact that this polarity causes vasoconstriction. Vasoconstriction effects the nerve endings which we perceive as pain. By adding a very small percentage of a histamine to the positive applicator, the pain can be offset because the histamine is a vasodilator. In accordance with the invention, this facilitates increasing the electrical current. For example, if the vasodilators of acetylcholine (ACh) or methacholine (MCh) are added to the solution on the positive applicator, it will be infused along with any other elements within the solution thereby lessening the pain sensation and allowing for an increase of current.

In addition to the large electrical current capability of this invention, amplification also contributes to an enormous signal to significantly increase drug delivery. Subsequently described in this application is a competitive environment of two different charge levels in solution that acts to recycle and thus amplify. Drug delivery of insulin can also be greatly enhanced with the use of a small quantity of glucose (perhaps 0.2 mol/l) in solution with insulin. The insulin infusion could be increased about 9 times with this glucose additive. Conversely, a small quantity of insulin added to the glucose withdrawal solution will increase the withdrawn analyte sample in a reverse iontophoresis modality. Still another method of increasing flux or signal amplification in drug delivery is with the use of charged chemicals as an integral part of a support structure or in a solution that communicates between skin and electrode. When two differently charged substances are in solution, they become reagents, thereby causing recycling resulting in amplification of the delivered drug. The use of negatively or positively charged insulin (charge depends on pH) in an environment of a positively charged membrane and using a negative power source to drive this solution into the skin, also increases amplification.

The aforedescribed systems describe various applications of charged molecules in either chemical form or as an integral part of a membrane, felt or equivalent support material. These applications include use of the polarized characteristic as a stand-alone infusion or delivery system, use of the polarized property to control bacterial or fungal infection and to promote healing, use of the charged property as a filter to stop movement of injury causing chemicals coming from tobacco and the like, use of the polarized characteristic to stop injurious chemicals coming from a battery or power supply in addition to various other applications. The use of charged chemicals or membranes or the like modified with these chemicals or the equivalent carrier of these chemicals is so unique and widely diverse that the present invention is directed to and includes the universality of use other than those limited prior uses by the aforementioned Pall Corporation for use in lab assays and industrial or clean air filters, but not for medical devices or personal care products and uses to enhance delivery of a beneficial flux, signal, or current.

### Non-Invasive Diagnostic Device

A major aspect this invention is the ultimate application of the use of chemically charged membranes or charged chemicals in liquid form making possible the first non-invasive rapid diagnostic test for glucose or other analytes. The finished device has a number of other innovations for its successful operability.

To better understand the details of these features and their contribution to the complete working unit, an overview of the elements and workings of the device is presented. While the device can be worn as a watch on the wrist or placed in other areas, it is perhaps easier to follow in the form of a probe because the active elements are stacked sequentially in the probe and can be more readily understood in that form.

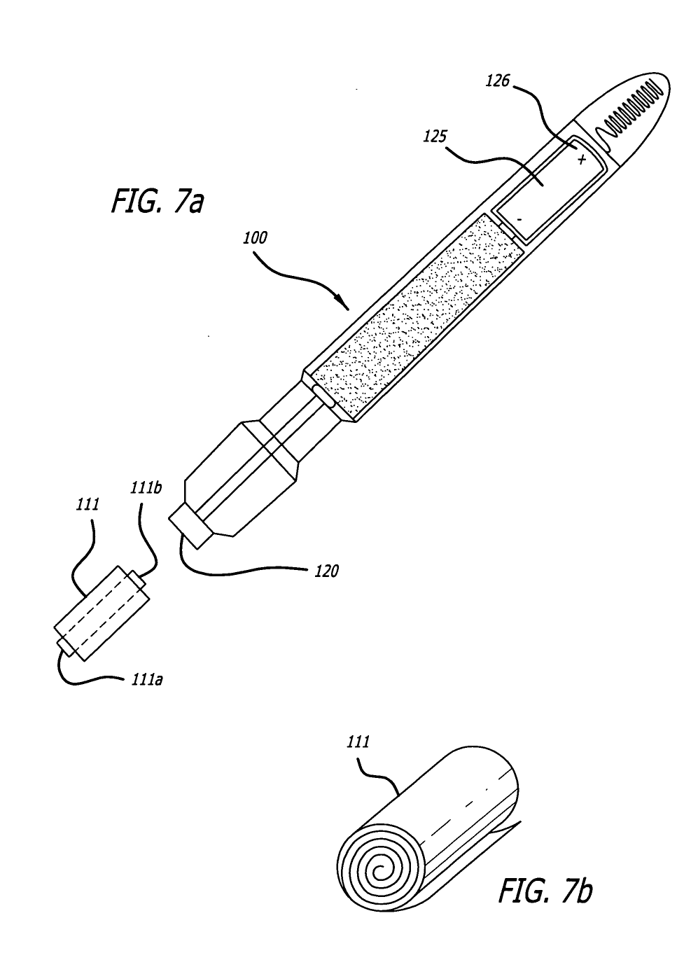

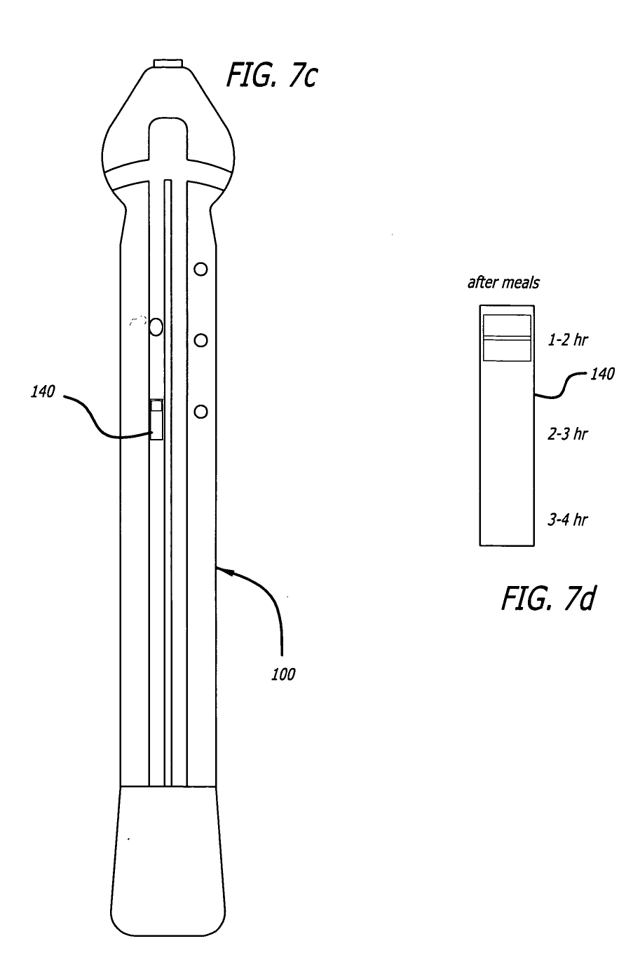

As shown in FIG. 7 the working probe is held as a pen 100 and placed on top of a vein on the wrist and makes an adequate glucose withdrawal within 95 seconds. The element touching the skin is a rolled, chemically charged membrane 111 that is wetted with a unique solution, gel or equivalent. The other end 111b of the membrane is in direct electrical contact with the negative terminal 120 of a power supply (battery) 125. There is now continuous electrical communication between the membrane end 111a that touches the skin and the negative terminal 120 because of the conductive, charged membrane 111. The circuit is complete when the patient is connected to the positive terminal 126 of the power source. The return electrode may also be placed on the forearm above of the wrist where a sample is taken. Another arrangement may be the grounding of the metal tube that is held by the hand applying the device to the wrist vein. The return electrode may also benefit from the use of charged membranes to prevent the hydrochloric acid generated at the positive electrode from reaching the skin. There are additional benefits with the use of the charged membranes. The charged membranes 111 are wetted with distilled deionized water and are conductive to the skin because the charged or ionic membranes are in direct contact with the skin. This eliminates the traditional saline wetted return electrode. It has been determined that the saline actually interfered with the withdrawn glucose sample giving distorted information. While the system described is intended to withdraw neutral or zwitterionic species by convective flow with the negative polarity to measure such clinically important substances as glucose, cholesterol, lactate etc., it has other uses without departing from the spirit and scope of the invention.

The invention may also be used to monitor drug pharmacokinetics by non-invasive electromigration withdrawal with the use of an opposite polarity electrode.

Each element of the diagnostic probe will now be explained to further disclose the unique features of the invention and how each element contributes to a working device.

CHEMICALLY CHARGED MEMBRANE(S) or FELT PADS—This is the building block of the device 100. Its multi-functions include: the need to stop the sodium hydroxide (lye) coming from the negative battery terminal. If not stopped, the sodium hydroxide would cause skin injury resulting in permanent scarring. This is prevented when a positively charged membrane is the intervenor between the negative terminal and the skin. The negative sodium hydroxide ions are attracted to the positively charged membrane and stopped from migrating to the skin. Meanwhile, the membrane(s) 111 allow the transport in the opposite direction of the withdrawn interstitial fluid containing glucose to travel to the negative electrode 120. This is aided by the fact that the surface of the negative electrode has a very high pH (because of the sodium hydroxide emitted there) that attracts glucose. It is essential that the inherent, extreme pH perform its function of glucose attraction. The charged membrane intervenor allows this to happen but prevents the skin-damaging sodium hydroxide from reaching the skin. The opposite end 111a of this membrane that touches the skin is maintained at a near neutral pH for safety. The glucose analyte to be measured accumulates at the end 111a of the membrane 111 touching the negative electrode 120. Accordingly, the probe 100 is designed with the inside end of the membrane removable from the rest of the probe. After making the withdrawal from the skin, the patient separates this piece from the probe and affixes it to the strip on a glucose monitor (not shown). The deposition of the withdrawn glucose as stated above is uniquely on the inside end 111b of the membrane 111. This point is approximately a half-inch away from the pickup end 111a of the membrane 111 that touches the skin. It is this inside part of the membrane that is placed on the strip of a commercially available monitor that will respond with a glucose reading. This part 111 is then discarded and replaced with a new unit.

Other key elements in this application of charged membranes is how they are presented to the withdrawn analyte. It is important that the membranes offer a continuous, unbroken path for the glucose to travel to the electrode. Any impediment to this flow results in inaccurate readings. For instance, early investigation made use of membranes positioned flat on the skin pickup area. Since it was essential to increase thickness of the charged membrane between the skin and the high voltage electrode to prevent skin injury and pain, experiments were conducted with layered membranes. Up to 75 and more layers may be necessary to prevent injury and allow enormous current density to speed treatment. Unfortunately, the layered membranes gave false readings because each layer presented an interface to the signal that distorted the readings. A roll of the charged membrane is now used that when positioned on its side, allows the signal to traverse a single surface on its way to the electrode. Increasing the height of the charged membrane increases protection and allows greater current. A half-inch membrane spacing between the skin and electrode may be used, but a greater spacing using a membrane of greater height may provide enhanced results. For instance, literature sets iontophoretic currents at 0.5 ma per cm² for safety and comfort. With the arrangement just described, as much as 31 times more current density could be drawn. The membranes used for this research and development are, again, a product of Pall Corporation with a fixed and limited concentration of charged chemicals as an integral part of the membranes. They were intended for purposes other than those described here in connection with the present invention. Membranes may be improved and its effects more pronounced by using membranes made with charged chemicals of a much higher concentration.

An alternate configuration to the above design is based on the following performance needs: It has been determined that reducing the conductance of the solution is paramount to performance. One design feature toward this end is the use of neutral glucose as a solution. Another would be for the conductive charged membrane that connects the skin to the wire screen electrode to be made less conductive. To accomplish this a second, nonconductive membrane is introduced that is connected with the conductive charged membrane whose other end touches the electrode. The nonconductive membrane is a thin (typically, about 0.005 inch) membrane that is placed on the end of the charged membrane and comes in direct contact with the skin. In this manner the electrical conductivity of the path between the skin and the electrode is reduced compared to the previous disclosure of a conductive membrane only. With this arrangement, the glucose formulation is also reduced to the vicinity of 250 mg/dl thus further reducing clutter. These steps improve accuracy.

SOLUTION—The all-important linkage between the active elements to make them function, is the solution. Conductivity between the electrode and the skin, even with a nonconductive solution, is made by virtue of the wetted charged or ionic membrane(s) that is in contact with the electrode and the skin. The formulation for glucose withdrawal should not add clutter that would compete with and impede this extremely small signal. To meet these requirements, glucose (solute) is provided in a distilled deionized water solvent to fulfill these needs and add other essential characteristics. For a formulation to work optimally, it is critical that the solution be saturated with glucose. If not totally saturated, then the water solvent would absorb the glucose signal and none would be available for analysis. Formulations used in the practice of the present invention provide for a glucose reading of 1 mg/dl or higher. This assures a saturated solution. Present formulations use a solute of glucose of 360 or higher mg/dl glucose in a solvent of distilled deionized water. Since analysis is made using the Medisense Precision QID instrument, this high level glucose solution does not affect the reading because the QID strip is unresponsive to this solution. Yet the withdrawn interstitial glucose fluid is processed as a blood sample and would be as reflected with a meter reading. Still another formulation for the solution would include a small quantity of insulin, perhaps 0.3% or less, in solution with the glucose. This combination has been found to greatly increase the signal passing through the membrane 111 on the way to the electrode 120, thus increasing amplification as subsequently described.

GLUCOSE AMPLIFICATION—In the subject invention, extremely low levels of glucose are electroosmotically withdrawn from the unbroken skin. It is very important that this small analyte (subpicomole level) be amplified to effect a reading in the shortest possible time. A novel means of doing this is still another use of the aforementioned chemically charged membranes 111 as a new form of reagent that results in chemical amplification of the glucose analyte with increased sensitivity and responsiveness. The reagent(s) is the charged chemical that is an integral part of a membrane. With the withdrawn glucose in solution passing through this membrane 111 drawn by the high pH negative terminal 120, the glucose reacts with self-replication. Factors that contribute to this amplification are the charged positive polarity of the membrane 111 in the negatively charged field. Another form of this amplification would be two or more adjacent membranes, one charged positively and another charged negatively. Glucose (or any other analyte) passing through these oppositely charged membranes would react by recycling and result in an amplified signal. Another form of this would be a membrane of one charge in an electric field of opposite charge. Another form would be two drugs or chemicals of different charges as previously mentioned. The competitive environment of the two different levels of charge in solution acts to recycle the glucose analyte. This ping-pong effect causing amplification allows analysis and quantification in the shortest possible time so that the reading is in real-time with the rapidly changing glucose in the body system. Increased amplification can be obtained using membranes made with a higher concentration of charged chemicals that will show increased reaction. This invention for chemical analysis lends itself to chip technology and in effect becomes ‘labs-on-a-chip’.

THREE POSITION CALIBRATION SWITCH—To cover the wide range of glucose readings necessary for health assessment, it is desirable to have a calibration switch 140 on the probe 100, as best shown in FIG. 7c). The switch is in conformity with the expected glucose changes as a result of food intake. Referring to FIG. 7d, this switch 140 allows an adjustment of the withdrawal time/current for the following criteria: Position 1, (1-2 hours after meals), Position 2 (2-3 hours after meals and Position 3 (3-4 hours after meals). This process of switch position selection at the time of use is so that the end result readings of the detection meter compare with the blood standard within allowable tolerances. By assigning different withdrawal time/current to expected different glucose levels, we are able to cover the widest possible range of readings. In experiments with the Precision QID strips, position 1 represented 1-2 hours after meals and was set at a dosimetry of time and current that related to the high glucose levels of the day. Position 2 related to approximately the midpoint between meals and was set at a dosimetry of time and current to represent this mid level. Position 3 represented the longest period away from the last meal and was set with a low dosimetry of time and current. Extended ranges may be reached when submultiple or multiples of the reading are employed to extend range. This factory adjustment can be used to extend the range beyond the present pursuit. Newer generation strips may require different electrical currents and time in each of these three switch positions to function properly. After the gross selections are made with the calibration switch, precise readings are obtained when the withdrawn glucose specimen is processed at the strip and results in a meter reading.

ACTIVE ELECTRODE—The electrode 120 is made of stainless steel, which is resistive to sodium hydroxide. Importantly, the electrode is typically a stainless steel screen. This was selected because the sodium hydroxide that is generated at this electrode would normally travel to the perimeter of a solid surface stainless steel electrode. This would create ‘hot spots’ and possibly cause distorted readings of the glucose containing membrane 111 in contact with the electrode 120. The use of the screen electrode causes many ‘perimeters’ and therefore contributes to uniform distribution of sodium hydroxide across its surface for more consistent readings. The power supply within the probe consists of a 6 volt battery with circuitry to increase the voltage to 70 volts. This powers the dosimetry circuitry with switch controls for the time and current of the output feeding the active electrode. The dosimetry circuit integrates time and current and terminates this supply at precisely the same value for every patient. Since every patient's resistance is a variable, this circuit will adjust itself time-wise to compensate for different individual's resistance while holding current constant, so that everyone is treated equally. Tapper U.S. Pat. Nos. 6,485,437 and 6,059,736 describe this in electrical circuitry detail.

While the above diabetes diagnostic device is described in detail as a probe 100, the technology could all be included in other forms. For instance, the commercial strip used in various monitors could have an additional piece attached to it that could include the membranes and be connected internally to the commercial monitor for power, dosimetry timer, etc. This extended strip piece could be applied to the skin for withdrawal and the withdrawal tip folded back over the enzyme sensitive target for reading. Another structure would be in the form of a watch with a rotary dial to select seven positions for up to seven readings a day. Each position would have its own membrane (which should be changed after each reading). The key membrane in play would be connected between the skin and the electrode as described for the probe 100 configuration.

It will be apparent from the foregoing description, that the new and improved system, method, apparatus and chemistry of the present invention satisfies the following features and other needs and objectives of the invention:

1) The use of charged chemicals of either negative or positive polarity on support members that could include a) membranes, b) felt pads made of natural or synthetic fibers, c) impregnated filter paper, d) liquid form, e) any material that allows charged chemicals to control ions, molecules or electrons;

2) The use of charged chemicals of either negative or positive polarity formulated with an increased concentration of the charged chemicals causing either negative or positive polarity to increase their effectiveness;

3) For use in a DC iontophoretic drug delivery system, the presence of charged chemicals in solution as an integral part of a felt pad(s) or a membrane(s) to prevent: a) injurious chemicals emanating from the electrode from reaching the skin, b) sodium hydroxide developed at the negative terminal is prevented from reaching the skin with either a negatively charged or positively charged intervenor between the skin and the electrode in an electrically conductive circuit, c) hydrochloric acid generated at the positive electrode can be prevented from reaching the skin with either positively charged or negatively charged chemicals on an appropriate support intervenor spaced between the electrode and the skin in an electrically conductive system;

4) The chemically charged intervenor(s) acting as a reservoir or storage area for the drug to be delivered;

5) In iontophoresis or reverse iontophoresis or drug delivery or similar chemical or drug transport system, the use of currents above the traditional 0.5 ma per cm². The aforementioned charged chemicals either on support structure or without support structure, enable these high currents to be achieved. Large molecular delivery also benefits from high electrical current along with Tapper U.S. Pat. Nos. 6,238,381 and 6,425,891;

6) In a powered patch, electrical currents can be further increased by a new physical configuration of the active, drug delivery applicator. High density current can be tolerated when multiple small circles of current emitting membranes are clustered instead of one large flat delivery surface;

7) The use of the aforementioned charged chemicals of both negative and positive polarity together or separately to meet all objectives of these inventions;

8) The use of charged chemicals on an intervenor or in combination with both polarities in an AC iontophoretic device;

9) The use of histamines in the positive applicator to lessen the pain from this applicator and allow an increase in current;

10) The use of charged membrane(s) in an otherwise unpowered patch (passive patch) to propel the drug(s) into the skin at greatly increased levels compared to other unpowered patches;

11) The use of charged membrane(s) in an unpowered patch as a storage area or reservoir for drugs.

12) The use of charged membrane(s) of either polarity or in combination to increase infusion in an otherwise unpowered patch;

13) The use of charged membranes between skin and output electrode as the conductive element when wetted with distilled water without the need of a saline solution;

14) The use in iontophoresis or reverse iontophoresis of charged membranes between the skin and output electrode as the conductive element when wetted with distilled water as a means of avoiding clutter from conductive chemicals that may be added to enhance transport;

15) The use of charged membrane(s) in a powered patch of either polarity or in combination as an intervenor to prevent skin injury;

16) The use of charged membrane(s) in a powered patch of either polarity or in combination to act as a drug storage area or reservoir;

17) The use of charged membrane(s) in a powered patch of either polarity or in combination as a reservoir and intervenor to increase currents above 0.5 ma per cm² without skin injury;

18) In drug delivery, a small amount of glucose (0.2 mol/l) in solution with insulin can be amplified 9 times in an unpowered environment using charged membranes;

19) Feature 18 wherein the above solution is in a powered environment will increase the signal many-fold;

20) Feature 19 in a powered environment with the use of polarized membranes to act both to prevent skin injury and further enhance amplification;

21) In drug delivery, any chemical or drug in a powered environment and stored in oppositely charged membrane(s) will cause amplification;

22) Another example of drug or chemical amplification is using the phenomena of oppositely charged drugs to create recycling and thus amplification would be with glucosamine and chondroitin. Glucosamine is positive and chondroitin is negative and the two in solution with the appropriately charged membranes and in an appropriately polarized field, would benefit from amplification;

23) The use of charged chemicals on filters in cigarettes positioned between the tobacco and the end held in the mouth to prevent the migration of deleterious tobacco chemicals from entering the mouth upon inhaling;

24) The use of charged chemicals of either polarity or in combination on impregnated filter paper or membranes as an intervenor between tobacco and mouth to prevent polarized harmful chemicals from reaching the mouth.

25) The use of a tobacco extract or flavor in combination with Items 23 and 24;

26) A charged membrane of the same polarity of the tobacco extract or flavor to propel the extract or flavor into the mouth;

27) The use of charged chemicals as an inherent part of a bandage or the like, to have an antibacterial effect when placed over a wound;

28) The use of a chemically charged bandage or the like that comes precoated with an antiseptic or the antiseptic is added later. The charged chemicals will drive the antiseptic into the wound continuously when wetted or in gel form for communication between all elements;

29) The use of a chemically charged bandage or the like to enhance and speed wound healing when wetted;

30) The use of a charged chemical of negative polarity in a toothpaste containing fluoride to infuse the fluoride below the tooth's surface and gum to prevent cavities and disease;

31) The use of a charged chemical of negative polarity as an integral part or coating of toothbrush bristles to cause the fluoride of a toothpaste to be driven or infused into the teeth or gums;

32) The use of charged chemicals of either polarity as an additive to a germ-killing mouthwash to infuse the antiseptic into the teeth and gums;

33) The use of a charged chemical of negative polarity to be used as an intervenor between a battery-powered iontophoretic toothbrush and the bristles to prevent injurious sodium hydroxide from the negative terminal of the battery from reaching the teeth, gums or mucous membrane;

34) A stent coated with either positive or negative chemicals or both to cause elution and prevent restenosis.

35) A stent with a coating of charged chemicals of either charge or both that will elute like-charged chemicals coated on top of the charged coating to prevent restenosis;

36) A stent with a coating of charged chemicals either positively or negatively charged or both integrated with like-charged chemicals that will be propelled or eluted from the surface to prevent restenosis;

37) A stent coated with quaternary amine, sulfonic acid or carboxyl acid or the equivalent to cause elution either with another chemical that will be driven beyond the surface or the charged coating alone to elute when surrounding tissue and body fluids come in contact;

38) For cosmetic application, the use of charged chemicals in liquid form as a spray to be applied over a moisturizer base or any other skin conditioner. This will drive a like-charged cosmetic ingredient or skin improvement product deeper into the skin than topical application;

39) The use of salicylic acid and/or its derivative in combination with a charged chemical to limit its travel beneath the skin and thus prevent irritation;

40) The use of positively charged salicylic acid in combination with negatively charged sulfonic acid or carboxyl acid or the like to bind to each other and thus limit migration of the salicylic acid beneath the epidermis;

41) The use of salicylic acid as a skin spray as part of a two component system wherein a negatively charged spray follows;

42) Salicylic acid's pH buffered from three to four to a nonirritating pH of approximately five and infused into the skin by a like-charged chemical;

43) The use of negatively charged sodium salicylate with negatively charged chemicals to infuse the sodium salicylate into the skin for beneficial effects. The sodium salicylate and the negatively charged chemicals may be in formulation or they may be in the form of a two component system whereby the sodium salicylate is applied first as perhaps a spray and followed by a spray of negatively charged chemicals;

44) For cosmetic and personal care application, the use of charged chemicals in formulation with like-charged skin improvement material. The repelled skin improvement materials will be infused deeper into the skin;

45) The use of the negative and positive charges in formulation to result in a neutral charge and then mixed with the salicylic acid to limit its penetration to the epidermis;

46) The neutral formulation of Item 45 to be used as a spray following the application of salicylic acid to limit its penetration;

47) The use of positively charged chemicals such as quaternary amine in solution with salicylic acid to limit the depth of its penetration to the epidermis;

48) The use of charged or neutral control chemicals in the form of a spray to be applied over the initial application of salicylic acid to limit its depth of penetration to the epidermis;

49) In a permanent hair remover (Tapper U.S. Pat. Nos. 6,094,594 and 6,206,869) wherein a depilatory is driven into the follicle by an iontophoretic device, the use of a charged intervenor between battery and skin for the purposes of: a) to use high currents to expedite treatment, b) to use chemically charged intervenor to prevent skin injury, c) to use the chemically charged intervenor as the storage or reservoir vehicle for the depilatory;

50) A non-invasive diagnostic withdrawal device using a charged or polarized membrane(s), one end of which is positioned to touch the skin and the other end touching the electrode;

51) An alternate construction of Item 50 is the use of two membranes, one charged or conductive and the other nonconductive in direct contact with each other and spaced between the skin and the electrode;

52) The use of a charged membrane and an uncharged membrane in a withdrawal system whereby the uncharged membrane is placed in contact with the skin and the charged membrane in contact with the electrode to complete the circuit.

53) The use of a wool felt nib such as from a marker pen as the intevenor between the skin and electrode to prevent the passage of sodium hydroxide from the negative electrode from passing to the skin. The wool nib may or may not be coated with charged chemicals;

54) The charged or polarized intervenor of Item 50 that is approximately a half-inch in length and placed between skin and electrode;

55) The membrane of Items 50-54 between the skin and the electrode to protect against skin injury;

56) Increasing the height of this membrane of Items 50, 41 and 55 allows the use of currents above 0.5 ma per cm²;

57) This charged membrane intervenor of Items 50, 54, 55 and 56 allows the use of high currents above the traditional 0.5 ma/cm²;

58) Increasing the level of charge of the aforementioned membrane(s) by higher concentrations of the polarized material improves the benefits cited above;

59) A positively charged wetted (gel) membrane prevents skin injury by stopping the sodium hydroxide ions emitted from the negative electrode in an. electrically conductive circuit;

60) The charged membrane interposed between skin and negative terminal also stores the solution (or gel) necessary to effect communication or current flow between skin and electrode;

61) The target withdrawn glucose analyte passes through the membrane to the end that touches the negative electrode and becomes the critical pick-up point for the glucose monitor to read. This pick-up point for the withdrawn glucose analyte is unique in that it is against the electrode and not at membrane entry point in contact with the skin;

62) The charged membrane is constructed in a rolled form so that one side of the membrane touches the skin and the other side touches the electrode. In other words, when viewed from the skin, the withdrawn analyte sees only straight line, unbroken surfaces while migrating to the electrode;

63) The membrane form of Item 62 that causes the withdrawn glucose analyte to flow in a straight line while migrating to the electrode;

64) The structure of Items 62 and 63 that causes the withdrawn glucose signal deposition on the end of the membrane in direct contact with the electrode;

65 The use of a high pH from the electrode that attracts the glucose to this point of signal deposition;

66) The structure of Features 62, 63, 64 and 65 wherein the membrane end in contact with the electrode is the pick-up point for the withdrawn glucose which is then placed in contact with the monitor's strip for analysis or direct reading;

67) The positive return electrode may also use charged membranes to prevent skin damage and allows toleration of higher currents;

68) The positive return electrode of the system described above may also be used to monitor drug pharmacokinetics;

69) A solution formulated with glucose in a solvent of distilled deionized water;

70) The solution of Item 69 wherein the glucose must totally saturate the distilled deionized water to prevent absorption of the withdrawn glucose;

71) The solution of Items 69 and 70 must have a surplus of glucose (distilled deionized water solvent totally saturated) with a monitor reading between 1 and 400 mg/dl or more;

72) Adding a small quantity of insulin to the glucose solutions of Items 69, 70 and 71, perhaps 0.3% or less, greatly increases the analyte signal or causes amplification;

73) The solution of Items 69, 70, 71 and 72 that may include stabilizers or preservatives;

74) Chemical amplification of the minute subpicomole withdrawn analyte takes place in the environment disclosed. When at least two differently charged substances occupy the same area, they become reagents to a signal passing through. The reagents cause recycling of the signal resulting in amplification. The charged positive membrane in the negative field acts as a reagent to the withdrawn glucose analyte and amplifies it;

75) Another form of this would be the use of a positive and negative membrane adjacent to each other to cause recycling and therefore amplification of analyte;

76) Amplification would take place if all the polarities cited above were reversed;

77) Charged membranes made with higher concentration of charged chemicals will show increased amplification;

78) More presence of charged membrane will increase the reaction and therefore increase amplification;

79) This invention lends itself for the new technology of ‘labs-on-a-chip’;

80) The electrode is a screen made of stainless steel to evenly disperse sodium hydroxide and pH;

81) The power supply consists of a 6 volt battery with circuitry to increase the DC output voltage to 70 volts. Note: voltage may be higher or lower. A safety circuit or fail-safe circuit is included;

82) The circuit includes a dosimetry circuit (Tapper patents) that precisely controls the analyte withdrawal quantity based on time and current;

83) This non-invasive diagnostic device includes a calibration switch;

84) A multi-position switch that selects the withdrawal time/current to match the highs, lows, and in-between time glucose levels of the patient caused by meals, physical exercise, or insulin dose;

85) A multiple position switch that selectively adjusts time and current to conform with well established periods of glucose change related to meal intake (also physical exercise and insulin dose);

86) The switch of Items 84 and 85 for following selections: Position 1 (1-2 hours after meals), Position 2 (2-3 hours after meals), and Position 3 (3-4 hours after meals);

87) Different time/current rates are assigned to each of the three switch positions;

88) A submultiple or multiple of the meter reading to extend range;

89) After gross selection is made with switch according to Items 83, 84, 85, 86, 87 and 88a precise reading is obtained when the withdrawn glucose specimen is processed at the strip and results in a meter reading based on the glucose concentration or density in the withdrawn interstitial fluid; and

90) An LED to indicate the precise end to the withdrawal process.

It will be apparent from the foregoing that, while particular forms of the invention have been illustrated and described, various alternatives, modifications and variations can be made without departing from the spirit and scope of the invention. Accordingly, the invention is intended to embrace all such alternatives, modifications and variations and it is not intended that the invention be limited, except as by the following claims.

1. A flow control system, comprising:
a support member; and
charged chemicals of either negative and/or positive polarity on said support member; whereby said charged chemicals control the flow of ions, molecules or electrons.

2. A flow control system as recited in claim 1, wherein said support member includes membranes.

3. A flow control system as recited in claim 1, wherein said support member includes felt pads made of natural or synthetic fibers.

4. A flow control system as recited in claim 1, wherein said support member includes impregnated filter paper.

5. A flow control system as recited in claim 1, wherein said support member includes a liquid or gel form.

6. A flow control system as recited in claim 1, wherein said support member includes any materials that control the flow of ions, molecules or electrons.

7. A system as set forth in claim 1, wherein said charged chemicals of either negative and/or positive polarity are formulated with an increased concentration of the charged chemicals causing either negative or positive polarity to increase their effectiveness.

8. In an iontophoretic drug delivery system, the combination comprising: an electrode; charged chemicals in solution as an integral part of a support member such as felt pad(s) or a membrane(s) for prevention of a) injurious chemicals emanating from said electrode from reaching the skin, b) sodium hydroxide developed at the negative terminal from reaching the skin with either a negatively charged or positively charged intervenor between the skin and said electrode in an electrically conductive circuit, c) hydrochloric acid generated at the positive electrode terminal from reaching the skin, with either positively charged or negatively charged chemicals on an appropriate support intervenor spaced between the electrode and the skin in an electrically conductive system.

9. A fluid delivery patch for application to a biological subject, comprising:
a chemical to be delivered to the subject;
at least one charged membrane incorporating or adjacent said chemical for electrically driving said chemical into the subject;
and support means for supporting said membrane and said chemical in a proximity to the subject suitable for proper chemical delivery to the subject.

10. A fluid delivery patch as recited in any of claims 1-9 and further including:
additional electrical supply means to further enhance said charged membrane.

11. A system as recited in claim 8 wherein said chemically charged interventor(s) act as a reservoir or storage area for the drug to be delivered.

12. In a system as recited in any of claims 1-11, the use of electrical currents greater than the traditional 0.5 ma per cm.

13. Apparatus adapted for placement within a biological subject, comprising:
a stent; and
a coating of electrically charged chemicals deposited on said stent.

14. Apparatus as recited in claim 13, wherein said chemicals may be either positive or negative or both to cause elution and prevent restenosis.

15. In an AC iontophoretic device, the use of charged chemicals on an intervenor or in combination with both positive and negative polarities.

16. In an iontophoresis or reverse iontophoresis system, the combination comprising:
electrically charged membranes between the skin, and an output electrode as the conductive element when wetted with distilled water, whereby clutter from conductive chemicals that may be added to enhance transport is avoided.

17. In a drug delivery system, the use of a small amount of glucose in solution with insulin for substantial amplification in a powered environment using charged membranes.

18. In an improved cigarette filter: the use of charged chemicals on the filters positioned between the tobacco and the end held in the mouth of a smoker, to prevent the migration of deleterious tobacco chemicals from entering the mouth upon inhaling.

19. An improved cigarette filter as recited in claim 18, including the use of charged chemicals of either polarity or in combination on impregnated filter paper or membranes, as an intervenor between tobacco and mouth to prevent polarized harmful chemicals from reaching the mouth.

20. In a medical application, the use of a chemically charged bandage or the like to enhance and speed wound healing.

21. For dental applications, the use of a charged chemical of negative polarity to infuse the fluoride in a toothpaste below the tooth's surface and gum to prevent cavities and disease.

22. For use in cosmetic and personal care applications, the method comprising:
use of charged chemicals in liquid form as a spray to be applied over a moisturizer base or any other skin conditioner.

23. A new and improved method, comprising:
using positively charged salicylic acid in combination with negatively charged sulfonic acid or carboxyl acid or the like to bind to each other and thus limit migration of the salicylic acid beneath the epidermis;

24. A new and improved method, comprising:
use of salicylic acid as a skin spray as part of a two component system wherein a negatively charged spray follows.

25. A method for cosmetic and personal care applications, comprising:
the use of charged chemicals in formulation with like-charged skin improvement material, whereby the repelled skin improvement materials will be infused deeper into the skin.

26. A non-invasive diagnostic withdrawal device using a charged or polarized membrane(s), one end of which is positioned to touch the skin and the other end touching an electrode, whereby withdrawal is enhanced and skin injury is mitigated.

27. A withdrawal device as recited in claim 26, wherein the height of said membrane is increased to facilitate the use of currents above 0.5 ma per cm².

28. A withdrawal device as recited in claim 26, and further including:
a positively charged wetted membrane to prevent skin injury by stopping the sodium hydroxide ions emitted from the negative electrode in an electrically conductive circuit.

29. A device as set forth in claim 26, wherein target withdrawn glucose analyte passes through said membrane to the end that touches a negative electrode which becomes the critical pick-up point for a glucose monitor to read.

30. A device as set forth in claim 26, wherein said charged membrane is constructed in a rolled form so that one end of the rolled membrane touches the skin and the other end touches said electrode, whereby the withdrawn analyte sees only straight line, unbroken surfaces while migrating to said electrode.

31. A device as recited in any of claims 26-30, including provision of a high pH from the electrode that attracts the glucose to the point of signal deposition.

32. A device as recited in claim 31, wherein the membrane end in contact with said electrode is the pick-up point for the withdrawn glucose which is then placed in contact with a monitor's strip for analysis or direct reading.

33. A device as recited in claim 26, wherein said electrode includes a screen made of stainless steel to evenly disperse sodium hydroxide and pH.

34. A device as recited in claim 26, wherein a dosimetry circuit is provided that precisely controls the analyte withdrawal quantity based on time and current.

35. A device as recited in claim 26, and further including: a calibration switch in the form of a multi-position switch that selects the withdrawal time/current to match the highs, lows, and in-between time glucose levels of a patient caused by meals, physical exercise, or insulin dose.

36. A multi-position switch as recited in claim 35, having the following selections: Position 1 (1-2 hours after meals), Position 2 (2-3 hours after meals), and Position 3 (3-4 hours after meals).

37. In an apparatus as recited in either of claims 35 or 36, the improvement comprising:
readout means for obtaining a precise reading when the withdrawn glucose specimen is processed at a strip and provides a readout based on the glucose concentration or density in the withdrawn interstitial fluid.

38. A new and improved method, comprising:
mixing a positively charged chemical with an antiperspirant and
applying the mixture to a biological subject,
whereby said charged chemical will drive said antiperspirant deep down the eccrine sweat duct for greater effectiveness than topical application.

39. A method as recited in claim 38, wherein said antiperspirant is aluminum chloride and the like.

40. A new and improved method, comprising:
applying to a subject a first spray containing a suitable antiperspirant;
following said first spray with a second spray of a positively charged chemical to infuse said antiperspirant deeply into said subject.

41. A method as recited in claim 40, wherein said antiperspirant is aluminum chloride and the like.

42. A new and improved method, comprising:
applying to a subject a first spray containing a suitable substance for infusion into the subject;
following said first spray with a second spray of a charged chemical to infuse said substance deeply into the subject.

43. A device as recited in claim 26 and further including:
the use of two membranes, one charged or conductive and the other nonconductive in direct contact with each other and spaced between the skin and the electrode.

44. A device as recited in claim 43, wherein said nonconductive membrane is placed in contact with the skin and said charged membrane in contact with the electrode to complete the circuit.

45. A device as recited in claim 26, and further including:
a wool felt nib as an intervenor between the skin and said electrode to prevent the passage of sodium hydroxide from the negative electrode to the skin.

46. A device as recited in claim 45 wherein said nib may be coated with charged chemicals.

47. Each and every novel feature and/or combination of features herein disclosed.
